(19) United States
(12) Patent Application Publication
(10) Pub. No.: US 2024/0157003 A1
(43) Pub. Date: May 16, 2024

(54) ULTRAVIOLET LIGHT IRRADIATION SYSTEM AND ULTRAVIOLET LIGHT IRRADIATION METHOD

(71) Applicant: NIPPON TELEGRAPH AND TELEPHONE CORPORATION, Tokyo (JP)

(72) Inventors: Ayako IWAKI, Musashino-shi, Tokyo (JP); Takahito KIRIHARA, Musashino-shi, Tokyo (JP); Tomohiro TANIGUCHI, Musashino-shi, Tokyo (JP); Satoshi NARIKAWA, Musashino-shi, Tokyo (JP); Kazuhide NAKAJIMA, Musashino-shi, Tokyo (JP); Takashi MATSUI, Musashino-shi, Tokyo (JP); Nobutomo HANZAWA, Musashino-shi, Tokyo (JP); Yuto SAGAE, Musashino-shi, Tokyo (JP); Chisato FUKAI, Musashino-shi, Tokyo (JP)

(73) Assignee: NIPPON TELEGRAPH AND TELEPHONE CORPORATION, Tokyo (JP)

(21) Appl. No.: 18/282,197

(22) PCT Filed: Apr. 5, 2021
(86) PCT No.: PCT/JP2021/014473

§ 371 Date: Sep. 14, 2023

(51) Int. Cl. A61L 2/10; A61L 2/24; A61L 2/26

(52) U.S. Cl. CPC A61L 2/10; A61L 2/24; A61L 2/26; A61L 2202/11; A61L 2202/14

(57) ABSTRACT

An object of the present invention is to provide an ultraviolet light irradiation system and an ultraviolet light irradiation method which can perceive a state of a region where ultraviolet light is irradiated to output/block the ultraviolet light.

An ultraviolet light irradiation system 301 according to the present invention includes an ultraviolet light source unit 11 that produces ultraviolet light,
an N (N is a natural number) irradiation unit 13 that irradiates an irradiation target region Ar with the ultraviolet light, a sensor unit 31 that detects whether or not an object that should avoid exposure is present in the irradiation target region Ar, and a blocking unit 30 that stops, in a case where the object that should avoid exposure is present in the irradiation target region Ar, irradiation of the ultraviolet light from the irradiation unit 13 to the irradiation target region Ar.

### TECHNICAL FIELD

The present disclosure relates to an ultraviolet light irradiation system and a decontamination method for performing sterilization and virus inactivation using ultraviolet light.

### BACKGROUND ART

For the purpose of preventing infectious diseases and the like, the demand grows for a system that uses ultraviolet light to perform sterilization and virus inactivation. In the present embodiment, the “decontamination” includes sterilization and virus inactivation.

There are three major categories of products in a decontamination system.

(1) Mobile Sterilization Robot

The mobile sterilization robot is an autonomous mobile robot that emits ultraviolet light. The mobile sterilization robot can automatically perform decontamination in a wide range without human intervention by emitting ultraviolet light while moving in a room in a building such as a hospital room. Refer to, for example, the website of Kantum Ushikata Co., Ltd. (https://www.kantum.co.jp/product/sakkin_robot/sakkinn_robo t/UVD robot).

(2) Stationary Air Purifier

The stationary air purifier is a device that is installed on a ceiling or at a predetermined place in a room and decontaminates air in the room while circulating the air. The stationary air purifier does not emit ultraviolet light to the outside and does not affect the human body, and thus highly safe decontamination can be performed. Refer to, for example, the website of Iwasaki Electric Co., Ltd. (https://www.iwasaki.co.jp/optics/sterilization/air/air03.h tml).

(3) Portable Sterilizer

The portable sterilizer is a portable device equipped with an ultraviolet light source such as a fluorescent lamp, a mercury lamp, or an LED. The user carries the portable sterilizer to an area where decontamination is to be performed, and irradiates the area with ultraviolet light. Thus, the portable sterilizer can be used in various places. Refer to, for example, the web site of Funakoshi Co., Ltd. (https://www.funakoshi.co.jp/contents/68182).

### CITATION LIST

### Non Patent Literature

Non Patent Literature 1: Baba et al., “Development of UV-Dedicated Heat-Resistant Optical Fibers (2)”, MITSUBISHI CABLE INDUSTRIES, LTD. Current News, No. 100, pp. 84-88, April 2003

### SUMMARY OF INVENTION

### Technical Problem

The conventional technologies have the following difficulties.
(1) Since the mobile sterilization robot applies high-output ultraviolet light, the device is large-scale and expensive. It is therefore difficult to realize the mobile sterilization robot economically.
(2) Since the stationary air purifier uses a method for sterilizing the circulated indoor air, it is difficult to immediately decontaminate clothes and the like, and bacteria and viruses emitted from carriers.
(3) The portable sterilizer has a problem that ultraviolet light to be emitted therefrom is relatively weak, which makes it difficult to perform decontamination in a short time. In addition, even if a high-output mercury lamp or a fluorescent lamp is used, the lamps are generally large and have a short life and light is diffused in proportion to the square of the distance to reduce the power; therefore, it is difficult to use the lamps in the portable sterilizer.

In order to address the problems (1) to (3), a system using an optical fiber is possible (refer to Non Patent Literature 1, for example). A thin and bendable optical fiber is used to transmit ultraviolet light from a light source, which provides flexibility to irradiate a specific place to be decontaminated with ultraviolet light outputted from a tip of the fiber. In addition, a P-MP system configuration used in the FTTH is adopted to share a single light source, so that economization can be expected.

It is to be noted that deep ultraviolet light used in a sanitization system using ultraviolet light causes a skin cancer or a cataract in a case where the deep ultraviolet light is applied to eyes and skin of living organisms including humans. Therefore, in a space where a person always stays, such as a living space, it is necessary to perform an operation of starting/stopping light output from a light source so as not to irradiate the person with ultraviolet light.

However, in the sanitization system described above, in a case where the location of the light source and an area where light is to be irradiated are not close to each other and a person enters the irradiated area, or, alternatively, in a case where ultraviolet light leaks due to breakage of an optical fiber connecting the light source and the irradiated area, or the like, the fact of the leakage cannot be perceived on the light source side, and the operation of stopping the light output cannot be performed, which possibly causes ultraviolet exposure to a person, for example.

That is, a conventional decontamination system using an optical fiber has a problem that it is difficult to perceive conditions that cause ultraviolet exposure and to block ultraviolet light.

In light of the above, in order to solve the problems described above, an object of the present invention is to provide an ultraviolet light irradiation system and an ultraviolet light irradiation method that allow to perceive a state of a region where ultraviolet light is irradiated to output/block the ultraviolet light.

### Solution to Problem

In order to achieve the above object, in an ultraviolet light irradiation system according to the present invention, a sensor unit checks a state of a region where ultraviolet light is irradiated to control output/blockage of the ultraviolet light.

Specifically, the ultraviolet light irradiation system according to the present invention includes:
an ultraviolet light source unit that produces ultraviolet light;
an N irradiation unit that irradiates a desired region with the ultraviolet light, where N is a natural number;
a sensor unit that detects whether or not an object that should avoid exposure is present in the desired region; and
a blocking unit that stops, in a case where the object that should avoid exposure is present in the desired region, irradiation of the ultraviolet light from the irradiation unit to the desired region.

The ultraviolet light irradiation method according to the present invention is an ultraviolet light irradiation method for an N irradiation unit to irradiate a desired region with ultraviolet light produced by an ultraviolet light source unit, where N is a natural number, and the ultraviolet light irradiation method includes:
detecting whether or not an object that should avoid exposure is present in the desired region; and
stopping, in a case where the object that should avoid exposure is present in the desired region, irradiation of the ultraviolet light from the irradiation unit to the desired region.

The present ultraviolet light irradiation system uses the sensor unit to check a state of a region where ultraviolet light is irradiated, blocks the ultraviolet light when an object (person or animal) that should avoid exposure to the ultraviolet light is detected, and outputs the ultraviolet light to the region where ultraviolet light is irradiated when the object that should avoid exposure is not detected. Therefore, according to the present invention, it is possible to provide an ultraviolet light irradiation system and an ultraviolet light irradiation method that allow to perceive a state of a region where ultraviolet light is irradiated to output/block the ultraviolet light.

For example, the blocking unit of the ultraviolet light irradiation system according to the present invention is an optical shutter that is disposed in an optical transmission line from the ultraviolet light source unit to the irradiation unit, is operable to close the optical transmission line in a case where the object that should avoid exposure is present in the desired region, and is operable to open the optical transmission line in a case where the object that should avoid exposure is not present in the desired region.

For example, the blocking unit of the ultraviolet light irradiation system according to the present invention is a light source control unit that causes the ultraviolet light source unit to stop outputting the ultraviolet light in a case where the object that should avoid exposure is present in the desired region, and causes the ultraviolet light source unit to output the ultraviolet light in a case where the object that should avoid exposure is not present in the desired region.

In a case where the blocking unit is the light source control unit, information from the sensor unit may be notified to the light source control unit via a path different from an optical transmission line from the ultraviolet light source unit to the irradiation unit.

In a case where the blocking unit is the light source control unit, information from the sensor unit may be notified to the light source control unit at a wavelength different from a wavelength of the ultraviolet light via an optical transmission line from the ultraviolet light source unit to the irradiation unit.

The ultraviolet light irradiation system according to the present invention may be configured to branch ultraviolet light and irradiate a plurality of regions where the ultraviolet light is irradiated. In the case of this configuration (in the case of N 2),
identification information is given to each of the sensor units,
the ultraviolet light source unit includes one or more light sources for supplying the ultraviolet light to each of the irradiation units, and
the light source control unit controls, based on the identification information, to output or stop outputting the ultraviolet light supplied by the light source.

The ultraviolet light irradiation system according to the present invention preferably further includes
on the ultraviolet light source unit side, a sensor information light source unit that supplies carrier light having a wavelength different from the wavelength of the ultraviolet light to the sensor unit side, and
on the sensor unit side, a light modulation unit that modulates the carrier light to generate information from the sensor unit and transmits the information to the light source control unit. The light source side supplies a carrier wave for an optical signal outputted from the sensor unit side, which eliminates the need for a light source on the sensor unit side, leading to reduction in power consumption accordingly.

Note that the inventions described above can be combined in any possible manner.

### Advantageous Effects of Invention

According to the present invention, it is possible to provide an ultraviolet light irradiation system and an ultraviolet light irradiation method that allow to perceive a state of a region where ultraviolet light is irradiated to output/block the ultraviolet light.

### BRIEF DESCRIPTION OF DRAWINGS

FIG. 1 is a diagram illustrating an ultraviolet light irradiation system according to the present invention.

FIG. 2 is a diagram illustrating a propagation section of the ultraviolet light irradiation system according to the present invention.

FIG. 3 is a diagram illustrating a cross-section of an optical fiber.

FIG. 4 is a diagram illustrating an ultraviolet light irradiation system according to the present invention.

FIG. 5 is a diagram illustrating an ultraviolet light irradiation system according to the present invention.

FIG. 6 is a diagram illustrating an ultraviolet light irradiation system according to the present invention.

FIG. 7 is a diagram illustrating an ultraviolet light source unit of an ultraviolet light irradiation system according to the present invention.

FIG. 8 is a diagram illustrating an ultraviolet light irradiation system according to the present invention.

FIG. 9 is a diagram illustrating an ultraviolet light irradiation system according to the present invention.

FIG. 10 is a diagram illustrating an ultraviolet light irradiation system according to the present invention.

FIG. 11 is a diagram illustrating an ultraviolet light irradiation method according to the present invention.

### DESCRIPTION OF EMBODIMENTS

Embodiments of the present invention will be described below, with reference to the accompanying drawings. The embodiments described below are examples of the present invention, and the present invention is not limited to the following embodiments. Note that constituent elements having the same reference numerals in the present specification and the drawings denote the same constituent elements.

(Gist of the Invention)

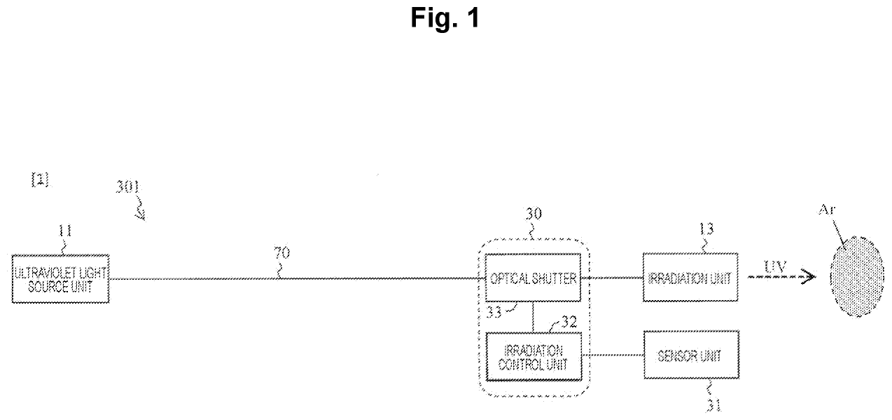

FIGS. 1 and 4 to 9 are diagrams illustrating an ultraviolet light irradiation system of the present invention.

The basic structure of the present system is a structure in which an ultraviolet light source unit 11 and an irradiation unit 13 for ultraviolet light are connected by an optical transmission line 70 and a sensor unit 31 is provided which detects the presence or absence of an object (person or animal) that should avoid irradiation of the ultraviolet light in an irradiation target region Ar.

In a case where the sensor unit 31 detects the object that should avoid irradiation in the irradiation target region Ar, the present system can prevent ultraviolet radiation exposure to the object that should avoid the exposure by stopping output of the ultraviolet light from the ultraviolet light source unit 11 or blocking the ultraviolet light propagated through the optical transmission line 70.

An exemplary structure of the present system will be described in detail below.

### First Embodiment

FIG. 1 is a diagram illustrating an ultraviolet light irradiation system 301 of the present embodiment. The ultraviolet light irradiation system 301 includes:
an ultraviolet light source unit 11 that produces ultraviolet light;
an N (N is a natural number) irradiation unit 13 that irradiates the irradiation target region Ar with the ultraviolet light;
a sensor unit 31 that detects whether or not an object that should avoid exposure is present in the irradiation target region Ar; and
a blocking unit 30 that stops, in a case where the object that should avoid exposure is present in the irradiation target region Ar, irradiation of the ultraviolet light from the irradiation unit 13 to the irradiation target region Ar.

The blocking unit 30 of the ultraviolet light irradiation system 301 is an optical shutter 33 that is disposed on the optical transmission line 70 from the ultraviolet light source unit 11 to the irradiation unit 13, is operable to close the optical transmission line in a case where the object that should avoid exposure is present in the irradiation target region Ar, and is operable to open the optical transmission line in a case where no object that should avoid exposure is present in the irradiation target region Ar.

The ultraviolet light is transmitted in the optical transmission line 70 to each irradiation unit 13. If the optical transmission line 70 is an optical fiber or an optical cable described below, the irradiation unit 13 can be laid in a small place where a robot or a device of conventional technologies cannot enter.

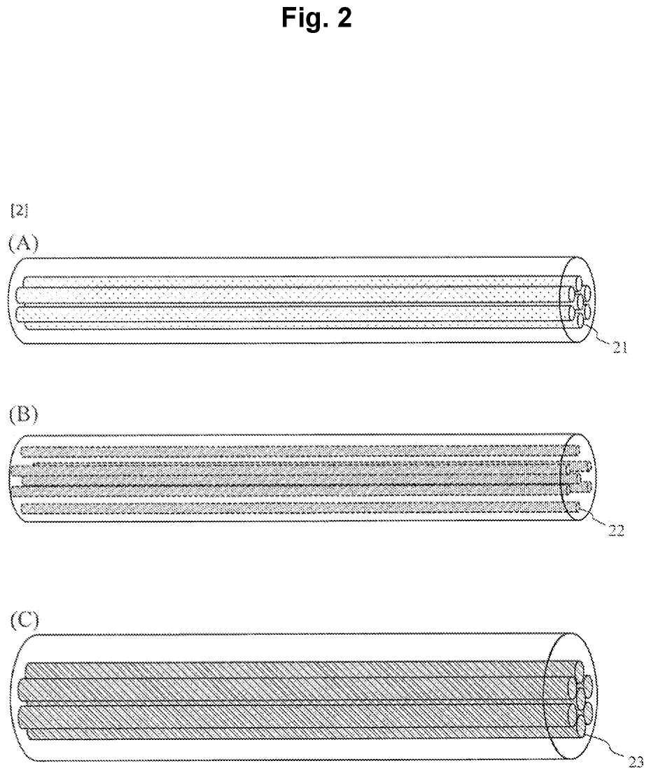

FIG. 2 is a diagram illustrating an optical cable or a multi-core optical fiber constituting the optical transmission line 70. FIG. 2(A) illustrates an optical cable in which a plurality of single-core optical fibers 21 is bundled. FIG. 2(B) illustrates a multi-core optical fiber having a plurality of cores 22. FIG. 2(C) illustrates an optical cable in which a plurality of multi-core optical fibers 23 is bundled.

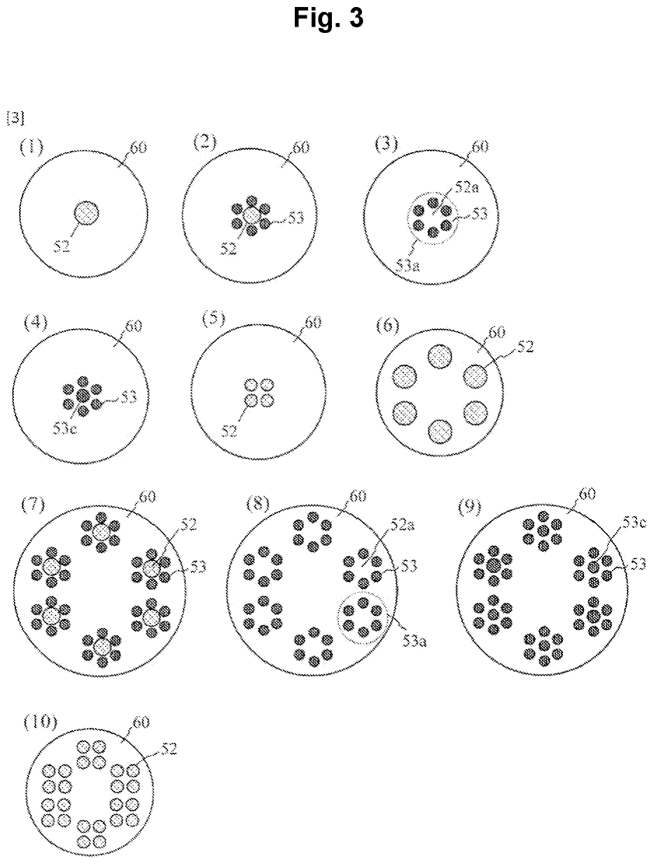

FIG. 3 is a diagram illustrating cross-sections of the single-core optical fiber and the multi-core optical fiber.

That is, the optical transmission line 70 can be an optical cable of the single-core optical fiber or the multi-core optical fiber, or can be a multi-core optical fiber illustrated in FIG. 3. The optical transmission line 70 may be a solid-type optical fiber using a general additive as illustrated in FIG. 3(1), an optical fiber having a hole structure illustrated in FIGS. 3(2) to 3(4), a multi-core optical fiber having a plurality of core regions illustrated in FIGS. 3(5) and 3(6), or an optical fiber (FIGS. 3(7) to 3(10)) having a combination of the structures of these optical fibers.

(1) Solid Core Optical Fiber

The optical fiber has, in cladding 60, one solid core 52 having a refractive index higher than that of the cladding 60. As used herein, the term “solid” means “not hollow”. The solid core can also be realized by forming an annular region having a low refractive index in the cladding.

(2) Hole-Assisted Optical Fiber

The optical fiber has, in cladding 60, a solid core 52 and a plurality of holes 53 arranged on the outer periphery thereof. The medium of the holes 53 is air, and the refractive index of the air is sufficiently lower than that of quartz-based glass. The hole-assisted optical fiber thus has a function of returning light leaked from the core 52 by bending or the like to the core 52 again, and has a small bending loss.

(3) Hole Structure Optical Fiber

The optical fiber has, in cladding 60, a hole group 53a including a plurality of holes 53, and has a refractive index effectively lower than that of a host material (glass or the like). This structure is called a photonic crystal fiber. The structure can have a structure including no high refractive index core having a changed refractive index, and it is possible to confine light by using a region 52a surrounded by the holes 53 as an effective core region. Compared with an optical fiber having a solid core, the photonic crystal fiber can reduce the influence of absorption and scattering loss due to additives in the core, and can realize optical characteristics that cannot be realized by a solid optical fiber, such as reduction of bending loss and control of a non-linear effect.
(4) Hollow Core Optical Fiber

In the optical fiber, a core region is formed of air. Light can be confined in the core region by using, in cladding region, a photonic band gap structure configured with a plurality of holes or an anti-resonance structure configured with a thin glass wire. The optical fiber has a small nonlinear effect, and can supply a high-power or high-energy laser.

(5) Coupling Core Type Optical Fiber

The optical fiber has, in cladding 60, a plurality of solid cores 52 having a high refractive index arranged close to each other. The optical fiber guides light between the solid cores 52 by optical wave coupling. Since the coupling core type optical fiber can disperse and transmit light by the number of cores, the power can be increased accordingly and efficient sterilization can be performed, and then the coupling core type optical fiber has an advantage that fiber degradation due to ultraviolet rays can be alleviated and the life can be extended.

(6) Solid Core Type Multi-core Optical Fiber

The optical fiber has, in cladding 60, a plurality of solid cores 52 having a high refractive index arranged apart from each other. The optical fiber guides light in a state where the influence of optical wave coupling can be ignored by sufficiently reducing the optical wave coupling between the solid cores 52. Therefore, the solid core type multi-core optical fiber has an advantage that each core can be treated as an independent waveguide.

(7) Hole-Assisted Type Multi-core Optical Fiber

The optical fiber has a structure in which a plurality of hole structures and the core regions of (2) described above are arranged in cladding 60.

(8) Hole Structure Type Multi-core Optical Fiber

The optical fiber has a structure in which a plurality of hole structures of (3) described above is arranged in cladding 60.

(9) Hollow Core Type Multi-core Optical Fiber

The optical fiber has a structure in which a plurality of hole structures of (4) described above is arranged in cladding 60.

(10) Coupling Core Type Multi-core Optical Fiber

The optical fiber has a structure in which a plurality of coupling core structures of (5) described above is arranged in cladding 60.

Note that the propagation mode in these optical fibers may be not only a single mode but also a multi-mode.

The ultraviolet light source unit 11 outputs ultraviolet light to the optical transmission line 70. The ultraviolet light source unit 11 may include one or more ultraviolet light sources. In a case where the optical transmission line 70 is an optical cable, the ultraviolet light source unit 11 inputs light to each optical fiber, and in a case where the optical transmission line 70 is a multi-core optical fiber, the ultraviolet light source unit 11 inputs light to each core.

The irradiation unit 13 irradiates a desired irradiation target region Ar with the ultraviolet light transmitted through the optical transmission line 70. The irradiation unit 13 includes an optical system such as a lens designed for a wavelength of an ultraviolet region.

The sensor unit 31 detects movement of an object (person, animal, or the like) that should avoid the irradiation around the irradiation target region Ar.

The blocking unit 30 of the ultraviolet light irradiation system 301 includes an irradiation control unit 32 and the optical shutter 33. In a case where an object that should avoid the irradiation is detected based on information received from the sensor unit 31, the irradiation control unit 32 blocks the ultraviolet light with the optical shutter 33 and causes the irradiation unit 13 to stop outputting the ultraviolet light. On the other hand, in a case where no object that should avoid the irradiation is detected based on information received from the sensor unit 31, the irradiation control unit 32 opens the optical shutter 33 and causes the irradiation unit 13 to start outputting the ultraviolet light.

The optical shutter 33 blocks or transmits the ultraviolet light propagating through the optical transmission line 70 based on a command from the irradiation control unit 32.

The ultraviolet light irradiation system 301 closes the optical shutter 33 and stops outputting the ultraviolet light to the irradiation target region Ar in a case where there is an object that should avoid the irradiation in the irradiation target region Ar, and opens the optical shutter 33 and restarts outputting the ultraviolet light to the irradiation target region Ar in a case where there is no object that should avoid the irradiation in the irradiation target region Ar. Thus, the ultraviolet light irradiation system 301 is capable of perceiving a state of a region where ultraviolet light is to be irradiated and outputting/blocking the ultraviolet light.

The ultraviolet light irradiation system 301 of FIG. 1 includes one ultraviolet light source unit 11 and one irradiation unit 13; however, another configuration is possible in which an optical distribution unit is provided in the optical transmission line 70, one ultraviolet light source unit 11 and N irradiation units 13 are provided, and the blocking unit 30 is disposed for each of the irradiation units 13.

### Second Embodiment

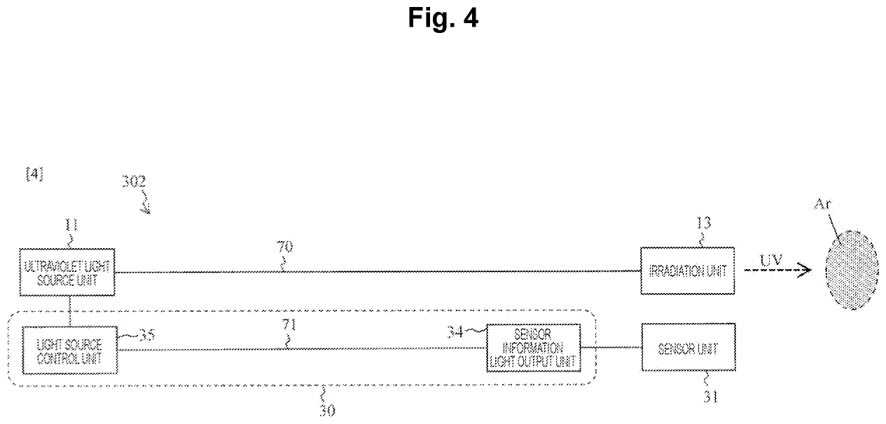

FIG. 4 is a diagram illustrating an ultraviolet light irradiation system 302 of the present embodiment. The ultraviolet light irradiation system 302 is different from the ultraviolet light irradiation system 301 of FIG. 1 in the configuration of the blocking unit 30. The blocking unit 30 of the ultraviolet light irradiation system 302 is a light source control unit 35 that causes the ultraviolet light source unit 11 to stop outputting the ultraviolet light in a case where the object that should avoid the irradiation is present in the irradiation target region Ar, and causes the ultraviolet light source unit 11 to output the ultraviolet light in a case where no object that should avoid the irradiation is present in the irradiation target region Ar.

In the present embodiment, information received from the sensor unit 13 is notified to the light source control unit 35 via a path different from the optical transmission line 70 from the ultraviolet light source unit 11 to the irradiation unit 13.

In the present embodiment, only configurations different from those of the ultraviolet light irradiation system 301 will be described.

The blocking unit 30 of the present embodiment includes a sensor information output unit 34, the light source control unit 35, and a signal line 71. The sensor information output unit 34 includes a transmitter, and transmits information detected by the sensor unit 31 to the signal line 71. The signal line 71 is an optical fiber, a metal wire, or wireless. In a case where the signal line 71 is an optical fiber, the signal line 71 may be an optical fiber or a core other than the optical fiber or the core that propagates the ultraviolet light of the optical transmission line 70. In a case where the signal line 71 is an optical fiber, the transmitter is an optical transmitter, and modulates carrier light based on information received from the sensor unit 31. The same applies to a case where the signal line 71 is a metal wire and a case where the signal line 71 is wireless. In a case where an object that should avoid the irradiation is detected based on the information received from the sensor unit 31 via the signal line 71, the light source control unit 35 controls the ultraviolet light source unit 11 to stop outputting the ultraviolet light. On the other hand, in a case where no object that should avoid the irradiation is detected based on the information received from the sensor unit 31 via the signal line 71, the light source control unit 35 controls the ultraviolet light source unit 11 to start outputting ultraviolet light.

The ultraviolet light irradiation system 302 stops outputting the ultraviolet light from the ultraviolet light source unit 11 in a case where there is an object that should avoid the irradiation in the irradiation target region Ar, and restarts outputting the ultraviolet light from the ultraviolet light source unit 11 in a case where there is no object that should avoid the irradiation in the irradiation target region Ar. Thus, the ultraviolet light irradiation system 302 is capable of perceiving a state of a region where ultraviolet light is to be irradiated and outputting/blocking the ultraviolet light.

### Third Embodiment

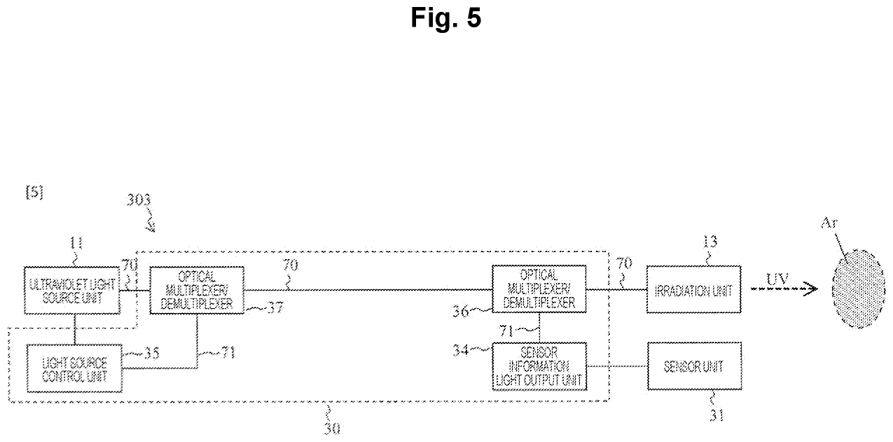

FIG. 5 is a diagram illustrating an ultraviolet light irradiation system 303 of the present embodiment. The ultraviolet light irradiation system 303 is different from the ultraviolet light irradiation system 301 of FIG. 1 in the configuration of the blocking unit 30. The blocking unit 30 of the ultraviolet light irradiation system 303 is a light source control unit 35 that causes the ultraviolet light source unit 11 to stop outputting the ultraviolet light in a case where the object that should avoid the irradiation is present in the irradiation target region Ar, and causes the ultraviolet light source unit 11 to output the ultraviolet light in a case where no object that should avoid the irradiation is present in the irradiation target region Ar.

In the present embodiment, information received from the sensor unit 31 is notified to the light source control unit 35 at a wavelength different from that of the ultraviolet light via the optical transmission line 70 from the ultraviolet light source unit 11 to the irradiation unit 13.

In the present embodiment, only configurations different from those of the ultraviolet light irradiation system 301 will be described.

The blocking unit 30 of the present embodiment includes the sensor information output unit 34, the light source control unit 35, an optical multiplexer/demultiplexer (36, 37), and a signal line 50.

The sensor information output unit 34 includes a transmitter, and transmits information detected by the sensor unit 31 to the signal line 71. The transmitter is an optical transmitter, and modulates carrier light based on information sent from the sensor unit 31. The wavelength of the carrier light is a wavelength that can be wavelength-division-multiplexed or demultiplexed with the ultraviolet light used for decontamination, and may be any wavelength as long as the optical transmitter can be configured. In the present embodiment, a case where the carrier light is infrared light will be described as an example.

The optical multiplexer/demultiplexer (36, 37) multiplexes/demultiplexes the infrared light for transmitting the sensor information received from the sensor information light output unit 34 into the optical transmission line 70 through which the ultraviolet light emitted from the irradiation unit 13 is propagated. Here, the ultraviolet light and the infrared light can be carried by the same optical fiber or the same core. In a case where the optical transmission line 70 is a multi-core optical fiber, the core for transmitting the ultraviolet light may be different from the core for transmitting the sensor information. In that case, the optical multiplexer/demultiplexer (36, 37) is a fan-in/fan-out device.

In a case where an object that should avoid the irradiation is detected based on the information received from the sensor unit 31 demultiplexed by the optical multiplexer/demultiplexer 37, the light source control unit 35 controls the ultraviolet light source unit 11 to stop outputting the ultraviolet light. On the other hand, in a case where no object that should avoid the irradiation is detected based on the information received from the sensor unit 31 demultiplexed by the optical multiplexer/demultiplexer 37, the light source control unit 35 controls the ultraviolet light source unit 11 to start outputting ultraviolet light.

The ultraviolet light irradiation system 303 stops outputting the ultraviolet light from the ultraviolet light source unit 11 in a case where there is an object that should avoid the irradiation in the irradiation target region Ar, and restarts outputting the ultraviolet light from the ultraviolet light source unit 11 in a case where there is no object that should avoid the irradiation in the irradiation target region Ar. Thus, the ultraviolet light irradiation system 303 is capable of perceiving a state of a region where ultraviolet light is to be irradiated and outputting/blocking the ultraviolet light.

### Fourth Embodiment

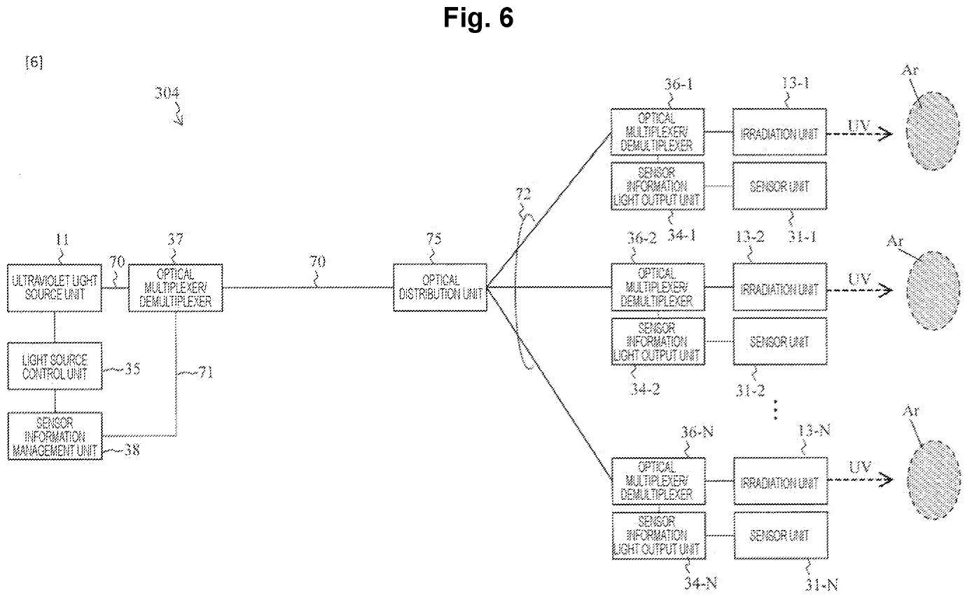

FIG. 6 is a diagram illustrating an ultraviolet light irradiation system 304 of the present embodiment. The ultraviolet light irradiation system 304 is different from the ultraviolet light irradiation system 303 of FIG. 4 in that a plurality of (N≥2) irradiation units 13 is provided.

In a case where N≥2 is satisfied,
identification information is given to each of the sensor units 13,
the ultraviolet light source unit 11 includes one or more light sources for supplying the ultraviolet light to each of the irradiation units 13, and
the light source control unit controls, based on the identification information, to output or stop outputting the ultraviolet light supplied by the light source.

In the present embodiment, only configurations different from those of the ultraviolet light irradiation system 303 will be described.

The optical transmission line 70 of the present embodiment is an optical cable in which a plurality of single-core optical fibers is bundled illustrated in FIG. 2 (A) or a multi-core optical fiber of FIG. 2 (B).

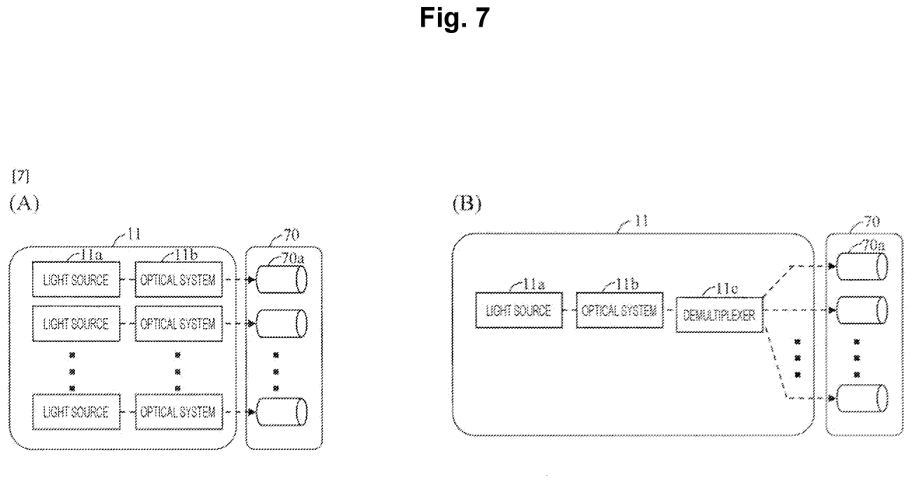

As illustrated in FIG. 7(A), the ultraviolet light source unit 11 includes a plurality of (N) light sources 11a. Ultraviolet light from each light source 11a is incident on the core 70a of the single-core optical fiber or each core 70a of the multi-core optical fiber of the optical transmission line 70 via an optical system 11b. As another configuration, the inside of the ultraviolet light source unit 11 may have a configuration as illustrated in FIG. 7 (B). Ultraviolet light from a single light source 11a is incident on the core 70a of the single-core optical fiber or each core 70a of the multi-core optical fiber of the optical transmission line 70 via the optical system 11b and a demultiplexer 11c. The light source 11a may have a configuration in which a plurality of light sources is arrayed inside to be used as one light source.

An optical distribution unit 75 distributes ultraviolet light transmitted from the ultraviolet light source unit 11 to a plurality of (N) single-core optical fibers 72. Specifically, the optical distribution unit 75 connects each optical fiber of the optical transmission line 70 or each core of the multi-core optical fiber to each of the single-core optical fibers 72 at a ratio of 1:1. That is, the light source 11a of the ultraviolet light source unit 11 and the irradiation target region Ar have a one-to-one relationship.

Sensor information light output units (34-1 to 34-N) have the following function in addition to the function of the sensor information light output unit 34 described above.

Each of the sensor information light output units (34-1 to 34-N) generates an optical signal for the sensor information at a wavelength different for each irradiation target region Ar, and transmits the optical signal to the ultraviolet light source unit 11 side by using either the optical fiber or the core of the optical transmission line 70 via an optical multiplexer/demultiplexer (36-1 to 36-N), the single-core optical fiber 72, and the optical distribution unit 75.

Alternatively, each of the sensor information light output units (34-1 to 34-N) generates an optical signal for the sensor information of each irradiation target region Ar at a wavelength (for example, infrared light) different from the wavelength of the ultraviolet light outputted from the irradiation unit 13, and transmits the optical signal to the ultraviolet light source unit 11 side by using the optical fiber or the core of the optical transmission line 70 corresponding to the irradiation target region Ar via the optical multiplexer/demultiplexer (36-1 to 36-N), the single-core optical fiber 72, and the optical distribution unit 75.

A sensor information management unit 38 is provided on the ultraviolet light source unit 11 side. The sensor information management unit 38 manages the surrounding situation (presence or absence of an object that should avoid the irradiation) in each irradiation target region Ar on the basis of the received sensor information and notifies the light source control unit 35 of the surrounding situation. The light source control unit 35 instructs the ultraviolet light source unit 11 to output ultraviolet light from the light source 11a corresponding to the irradiation target region Ar having no object that should avoid the irradiation and instructs the ultraviolet light source unit 11 not to output ultraviolet light from the light source 11a corresponding to the irradiation target region Ar having an object that should avoid the irradiation.

The ultraviolet light irradiation system 304 stops, for each irradiation target region Ar, outputting the ultraviolet light from the ultraviolet light source unit 11 in a case where there is an object that should avoid the irradiation, and restarts outputting the ultraviolet light from the ultraviolet light source unit 11 in a case where there is no object that should avoid the irradiation. Thus, the ultraviolet light irradiation system 304 is capable of perceiving a state of each region where ultraviolet light is to be irradiated and outputting/blocking ultraviolet light.

### Fifth Embodiment

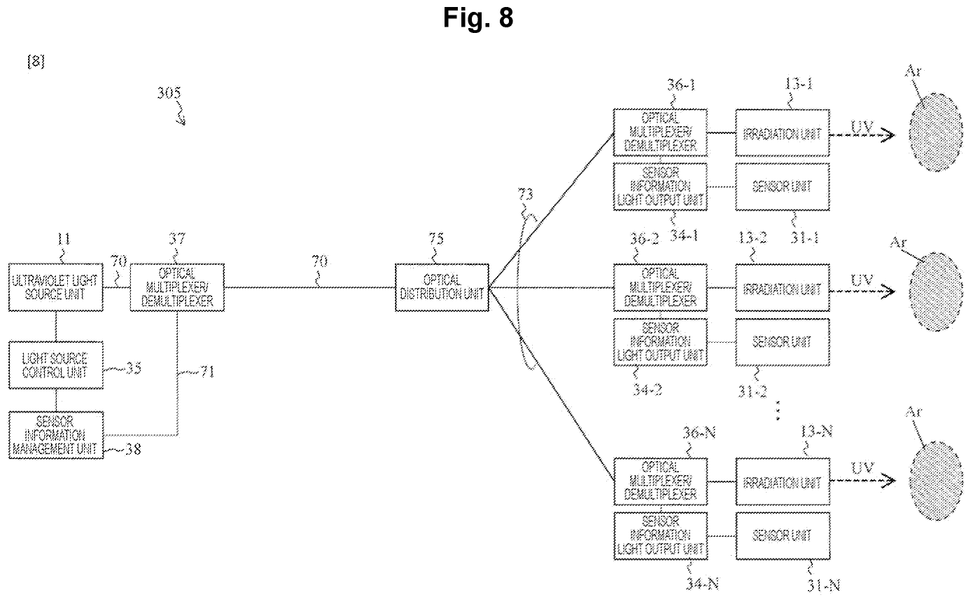

FIG. 8 is a diagram illustrating an ultraviolet light irradiation system 305 of the present embodiment. The ultraviolet light irradiation system 305 is different from the ultraviolet light irradiation system 304 of FIG. 6 in that the optical transmission line 70 is an optical cable in which a plurality of multi-core optical fibers is bundled illustrated in FIG. 2(C).

As illustrated in FIG. 7, the ultraviolet light source unit 11 includes a plurality of (N) light sources 11a. Ultraviolet light from each light source 11a is incident on the corresponding core 70a of the multi-core optical fiber of the optical transmission line 70 via the optical system 11b.

The optical distribution unit 75 distributes the ultraviolet light transmitted from the ultraviolet light source unit 11 to a plurality of (N) multi-core optical fibers 73. Specifically, the optical distribution unit 75 connects each multi-core optical fiber of the optical transmission line 70 to each of the multi-core optical fibers 73 at a ratio of 1:1. That is, the light source 11a of the ultraviolet light source unit 11 and the irradiation target region Ar have a one-to-one relationship.

The sensor information light output units (34-1 to 34-N) and the optical multiplexer/demultiplexer (36-1 to 36-N) are different from those of the ultraviolet light irradiation system 304 of FIG. 6 in the following points.

Each of the sensor information light output units (34-1 to 34-N) generates an optical signal for the sensor information for each irradiation target region Ar, and transmits the optical signal to the ultraviolet light source unit 11 side through the optical transmission line 70 via the optical multiplexer/demultiplexer (36-1 to 36-N), the multi-core optical fiber 73, and the optical distribution unit 75. At this time, the optical multiplexer/demultiplexer (36-1 to 36-N) inputs the optical signal for the sensor information to the core of the multi-core optical fiber 73 so as to satisfy the following two conditions in the optical transmission line 70.

(1) Other than the Core that Propagates the Ultraviolet Light to be Applied to the Irradiation Region Ar

(2) Different Cores for Each Irradiation Region Ar

If the foregoing conditions are satisfied, then the sensor information management unit 38 can identify which irradiation target region Ar corresponds to the sensor information.

The operation on the ultraviolet light source unit 11 side is similar to the operation on the ultraviolet light source unit 11 side of the ultraviolet light irradiation system 304 in FIG. 6.

The ultraviolet light irradiation system 305 also stops, for each irradiation target region Ar, outputting the ultraviolet light from the ultraviolet light source unit 11 in a case where there is an object that should avoid the irradiation, and restarts outputting the ultraviolet light from the ultraviolet light source unit 11 in a case where there is no object that should avoid the irradiation. Thus, the ultraviolet light irradiation system 305 is capable of perceiving a state of each region where ultraviolet light is to be irradiated and outputting/blocking ultraviolet light.

### Sixth Embodiment

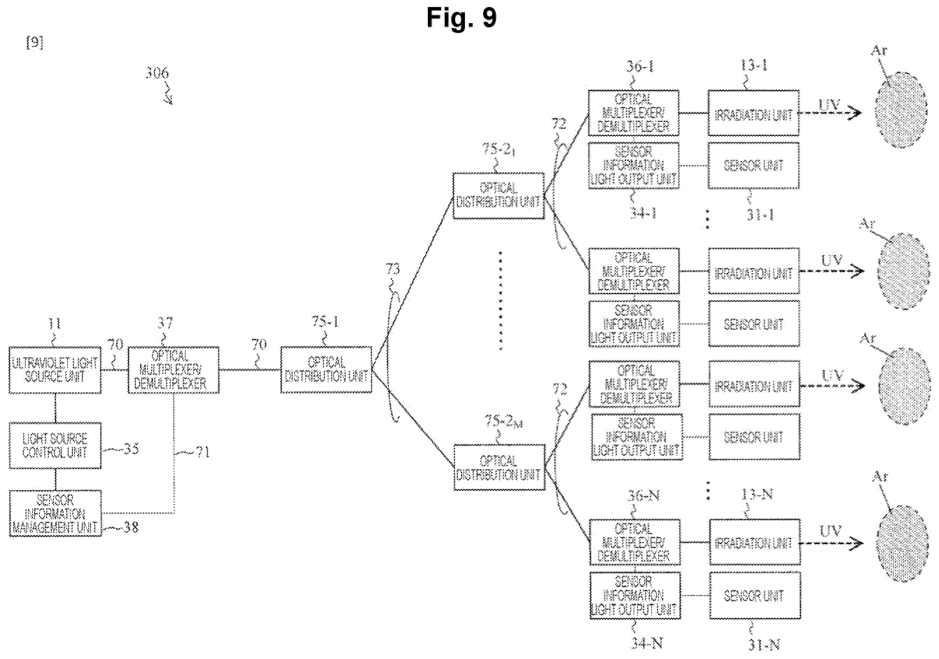

FIG. 9 is a diagram illustrating an ultraviolet light irradiation system 306 of the present embodiment. The ultraviolet light irradiation system 306 is different from the ultraviolet light irradiation system 305 of FIG. 8 in that the optical distribution units are configured in multiple stages.

The ultraviolet light irradiation system 306 includes one optical distribution unit 75-1 and M optical distribution units 75-2. The optical distribution unit 75-1 is the same as the optical distribution unit 75 included in the ultraviolet light irradiation system 305 of FIG. 8, and distributes the ultraviolet light transmitted from the ultraviolet light source unit 11 to a plurality of (M) multi-core optical fibers 73. Specifically, the optical distribution unit 75-1 connects each multi-core optical fiber of the optical transmission line 70 to each of the multi-core optical fibers 73 at a ratio of 1:1. The optical distribution unit 75-2 is the same as the optical distribution unit 75 included in the ultraviolet light irradiation system 304 of FIG. 6, and distributes the ultraviolet light transmitted through the multi-core optical fiber 73 to a plurality of (N) single-core optical fibers 72. Specifically, the optical distribution unit 75-2 connects each core of the multi-core optical fibers 73 to each of the single-core optical fibers 72 at a ratio of 1:1.

The optical transmission line 70 is an optical cable in which a plurality of multi-core optical fibers is bundled illustrated in FIG. 2(C). The optical fiber 72 can be an optical fiber having the structure described with reference to (1) to (5) of FIG. 3.

The functions of the sensor information light output units (34-1 to 34-N) and the sensor information management unit 38 are the same as the functions of the sensor information light output units (34-1 to 34-N) and the sensor information management unit 38 of the ultraviolet light irradiation system 304 of FIG. 6.

The ultraviolet light irradiation system 306 stops, for each irradiation target region Ar, outputting the ultraviolet light from the ultraviolet light source unit 11 in a case where there is an object that should avoid the irradiation, and restarts outputting the ultraviolet light from the ultraviolet light source unit 11 in a case where there is no object that should avoid the irradiation. Thus, the ultraviolet light irradiation system 306 is capable of perceiving a state of each region where ultraviolet light is to be irradiated and outputting/blocking ultraviolet light.

### Seventh Embodiment

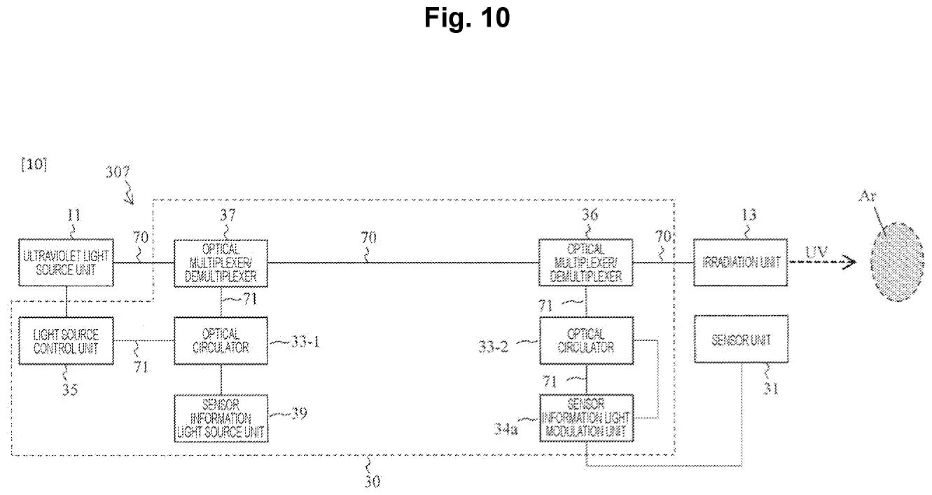

FIG. 10 is a diagram illustrating an ultraviolet light irradiation system 307 of the present embodiment. The ultraviolet light irradiation system 307 is different from the ultraviolet light irradiation system 303 of FIG. 5 in that the carrier light of the optical signal for the sensor information transmitted from the sensor side is supplied from the ultraviolet light source unit side.

Specifically, the ultraviolet light irradiation system 307 is configured to further include, in the ultraviolet light irradiation system 303 of FIG. 5:
on the ultraviolet light source unit 11 side, a sensor information light source unit 39 that supplies carrier light having a wavelength different from the wavelength of the ultraviolet light to the sensor unit 31 side, and
on the sensor unit side, in place of the sensor information output unit 34, a light modulation unit 34a that modulates the carrier light to generate sensor information from the sensor unit 31 and transmits the sensor information to the light source control unit 35.

Hereinafter, only differences from the ultraviolet light irradiation system 303 will be described. The sensor information light source unit 39 produces carrier light (continuous light) having a wavelength different from that of ultraviolet light (for example, infrared light). The carrier light passes through an optical circulator 33-1 and is multiplexed into the optical transmission line 70 by the optical multiplexer/demultiplexer 37. The carrier light may be multiplexed into the same core as the ultraviolet light in the optical transmission line 70, or may be multiplexed into a core or an optical fiber different from the ultraviolet light. The carrier light is demultiplexed from the optical transmission line 70 by the optical multiplexer/demultiplexer 36, passes through the signal line 71 and an optical circulator 33-2, and is supplied to a sensor information light modulation unit 34a. The sensor information light modulation unit 34a corresponds to the sensor information output unit 34 of the ultraviolet light irradiation system 303 of FIG. 5.

The sensor information light modulation unit 34a modulates the supplied carrier light based on the sensor information notified by the sensor unit 31, and outputs an optical signal for the sensor information to the signal line 71. The optical signal passes through the signal line 71 and the optical circulator 33-2 and is multiplexed into the optical transmission line 70 by the optical multiplexer/demultiplexer 36. The signal light may be multiplexed into the same core as the ultraviolet light in the optical transmission line 70, or may be multiplexed into a core or an optical fiber different from the ultraviolet light. Note that the signal light is multiplexed into a core or an optical fiber different from the carrier light. The signal light is demultiplexed from the optical transmission line 70 by the optical multiplexer/demultiplexer 37, passes through the signal line 71 and the optical circulator 33-1, and enters the light source control unit 35.

The operation of the light source control unit 35 is the same as that of the light source control unit 35 of the ultraviolet light irradiation system 303.

The ultraviolet light irradiation system 307 stops outputting the ultraviolet light from the ultraviolet light source unit 11 in a case where there is an object that should avoid the irradiation in the irradiation target region Ar, and restarts outputting the ultraviolet light from the ultraviolet light source unit 11 in a case where there is no object that should avoid the irradiation in the irradiation target region Ar. Thus, the ultraviolet light irradiation system 307 is capable of perceiving a state of a region where ultraviolet light is to be irradiated and outputting/blocking the ultraviolet light. Further, in the ultraviolet light irradiation system 307, it is not necessary to provide a light source on the sensor unit 31 side, and power consumption on the sensor unit 31 side can be reduced by the amount corresponding to the light source as compared with the case of the ultraviolet light irradiation system 303.

### Eighth Embodiment

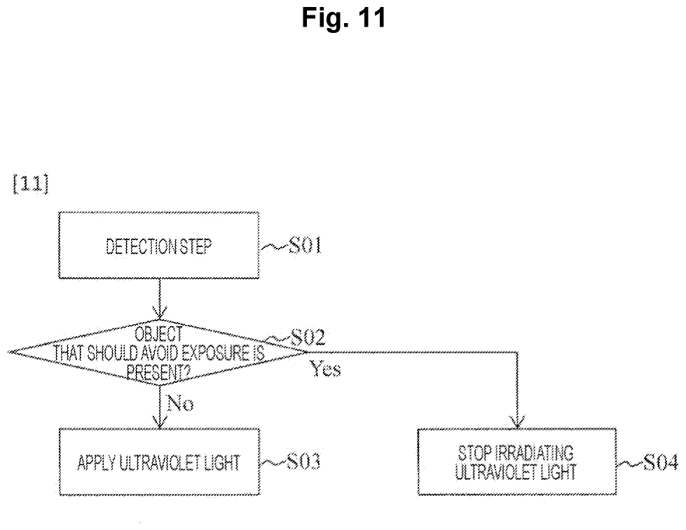

FIG. 11 is a flowchart illustrating an ultraviolet light irradiation method using the ultraviolet light irradiation system (301 to 307) of the present embodiment. The present ultraviolet light irradiation method is an ultraviolet light irradiation method for an N (N is a natural number) irradiation unit 13 to irradiate the irradiation target region Ar with ultraviolet light produced by the ultraviolet light source unit 11, and the method includes:
detecting whether or not an object that should avoid exposure is present in the irradiation target region Ar (step S01);
applying, in a case where the object that should avoid exposure is not present in the irradiation target region Ar (No in step S02), the ultraviolet light from the irradiation unit 13 to the irradiation target region Ar (step S03); and
stopping, in a case where the object that should avoid exposure is present in the irradiation target region Ar (Yes in step S02), irradiation of the ultraviolet light from the irradiation unit 13 to the irradiation target region Ar (step S04).

### REFERENCE SIGNS LIST

11 Ultraviolet light source unit
11a Light source
11b Optical system
13 Irradiation unit
21 Single-core optical fiber
22 Core
23 Multi-core optical fiber
Blocking unit
31 Sensor unit
32 Irradiation control unit
33 Optical shutter
33-1, 33-2 Optical circulator
34 Sensor information light output unit
34a Sensor information light modulation unit
35 Light source control unit
36, 37 Optical multiplexer/demultiplexer
38 Sensor information management unit
39 Sensor information light source unit
52 Solid core
52a Region
53 Hole
53a Hole group
60 cladding
70 Optical transmission line
71 Signal line
72 Single-core optical fiber
73 Multi-core optical fiber
75, 75-1, 75-2 Optical distribution unit
301 to 307 Ultraviolet light irradiation system

1. An ultraviolet light irradiation system comprising:
an ultraviolet light source unit that produces ultraviolet light;
an N irradiation unit that irradiates a desired region with the ultraviolet light, where N is a natural number;
a sensor unit that detects whether or not an object that should avoid exposure is present in the desired region; and
a blocking unit that stops, in a case where the object that should avoid exposure is present in the desired region, irradiation of the ultraviolet light from the irradiation unit to the desired region.

2. The ultraviolet light irradiation system according to claim 1, wherein the blocking unit is an optical shutter that is disposed in an optical transmission line from the ultraviolet light source unit to the irradiation unit, is operable to close the optical transmission line in a case where the object that should avoid exposure is present in the desired region, and is operable to open the optical transmission line in a case where the object that should avoid exposure is not present in the desired region.

3. The ultraviolet light irradiation system according to claim 1, wherein the blocking unit is a light source control unit that causes the ultraviolet light source unit to stop outputting the ultraviolet light in a case where the object that should avoid exposure is present in the desired region, and causes the ultraviolet light source unit to output the ultraviolet light in a case where the object that should avoid exposure is not present in the desired region.

4. The ultraviolet light irradiation system according to claim 3, wherein information from the sensor unit is notified to the light source control unit via a path different from an optical transmission line from the ultraviolet light source unit to the irradiation unit.

5. The ultraviolet light irradiation system according to claim 3, wherein information from the sensor unit is notified to the light source control unit at a wavelength different from a wavelength of the ultraviolet light via an optical transmission line from the ultraviolet light source unit to the irradiation unit.

6. The ultraviolet light irradiation system according to claim 3, wherein
in a case where N≥2 is satisfied,
identification information is given to each of the sensor units,
the ultraviolet light source unit includes one or more light sources for supplying the ultraviolet light to each of the irradiation units, and
the light source control unit controls, based on the identification information, to output or stop outputting the ultraviolet light supplied by the light source.

7. The ultraviolet light irradiation system according to claim 5, further comprising
on the ultraviolet light source unit side, a sensor information light source unit that supplies carrier light having a wavelength different from the wavelength of the ultraviolet light to the sensor unit side, and
on the sensor unit side, a light modulation unit that modulates the carrier light to generate information from the sensor unit and transmits the information to the light source control unit.

8. An ultraviolet light irradiation method for an N irradiation unit to irradiate a desired region with ultraviolet light produced by an ultraviolet light source unit, where N is a natural number, the ultraviolet light irradiation method comprising:
detecting whether or not an object that should avoid exposure is present in the desired region; and
stopping, in a case where the object that should avoid exposure is present in the desired region, irradiation of the ultraviolet light from the irradiation unit to the desired region.
